(19) United States
(12) Patent Application Publication
(10) Pub. No.: US 2024/0342331 A1
(43) Pub. Date: Oct. 17, 2024

(54) Method for Controlling Microbial Growth in Sugar Processing

(71) Applicant: HYDRITE CHEMICAL CO., Brookfield, WI (US)

(72) Inventors: Carl Schoenfelder, Lakeville, MN (US); Scott K. Cumming, Sun Prairie, WI (US)

(21) Appl. No.: 18/627,855

(22) Filed: Apr. 5, 2024

Related U.S. Application Data
(60) Provisional application No. 63/496,055, filed on Apr. 14, 2023.

(51) Int. Cl. A61L 2/18; A61L 2/24

(52) U.S. Cl. CPC A61L 2/186; A61L 2/24; A61L 2202/14

(57) ABSTRACT

A method for controlling microbial growth in a sugar processing system is disclosed. The method comprises adding a peroxy acid into water of an extraction system of the sugar processing system, wherein the extraction system extracts sugar from a sugar-containing plant material. The peroxy acid is added to the extraction system at one or more of the following addition points: water entering a diffuser of the extraction system, a mid-tower region of the extraction system, water entering the diffuser from a water supply, pressed pulp water from a pulp press of the extraction system, raw juice exiting a cossette mixer of the extraction system, juice recirculated from the diffuser to the cossette mixer, and a cossette slurry entering the cossette mixer. In one embodiment, the peroxy acid is peracetic acid.

### CROSS-REFERENCES TO RELATED APPLICATIONS

This application claims priority of U.S. Provisional Application Ser. No. 63/496,055 filed Apr. 14, 2023, and entitled “Method for Controlling Microbial Growth in Sugar Processing”, the contents of which is incorporated herein by reference in its entirety.

### STATEMENT REGARDING FEDERALLY SPONSORED RESEARCH

Not Applicable.

### BACKGROUND OF THE INVENTION

### 1. Field of the Invention

The invention relates to methods for controlling microbial growth in sugar processing.

### 2. Description of the Related Art

Sugar (sucrose) is primarily obtained from plant raw materials, such as sugar beets and sugar cane, by cutting the raw materials and extracting sugar-containing solutions from the plant parts. Sugar beets are subject to microbiological decay through bacteria, yeasts, and fungi within certain pH values. There is a risk of infestation by microorganisms during sugar processing. Microorganisms can degrade sugars contained in the raw materials and process materials to acids and gases to cause loss of sugar product, and/or cause elevated bacterial populations in the products. Microorganisms can influence the process negatively, not only by causing sugar losses, but also, for example, by causing pH drops and high lactic acid concentrations, which can affect other steps in the process.

A typical sugar beet processing operation includes a flume water system that is used to transport the beets from a post-harvest delivery or storage location into the factory beet washer while simultaneously removing field dirt that might adversely affect cutting and extraction. Acid production is a natural, continuous process in flume water due to acid-forming bacteria activity. To maintain an acceptable microbial count, a common method of flume water treatment is to maintain the pH in the alkaline range using lime. Maintaining this high pH controls acid forming bacterial activity.

Historic industry best practice regarding flume system water microbial management is to drive pH up to a range of 10.5 pH or above. This strategy is primarily driven by the rationale that a wide variety of bacteria species struggle to survive at such a high pH. By suppressing microbial activity in the flume, sugar recovery improves through the process.

The recognitions and detractions of this strategy using lime include:
i. The lime kiln and/or lime delivery system to the flume system must be robust, with adequate capacity for this strategy to be effective and efficient.
ii. There is a high relative cost with hydrated/pebble lime if/when kiln capacity cannot support flume water lime addition. Costs become greater in feeder systems where the pebble lime is not fully dissolved.
iii. If pH decreases below a certain point, an inordinate amount of lime is required to reach or regain the high pH, at high input costs.
iv. Hard calcium scale develops repeatedly over time, blinding screens and other equipment, typically resulting in a deliberate manipulation of flume system pH downward. While pH is suppressed to break down scale, bacteria takes hold which contribute to sugar losses, and once again, an inordinate amount of lime is required to regain control.
v. Inter-campaign maintenance costs associated with flume system de-scaling can be high.

Sugar processing plants that do not have sufficient kiln capacity or necessary lime handling equipment to effectively maintain a high pH strategy have compounded issues associated with low pH (>4 but <10) flume water, such as:
i. excessive lime is expended for limited, and sometimes negligible return on investment;
ii. accelerated flume system equipment corrosion occurs; and
iii. high bacteria loading carrying forward to the process results in excessive sugar losses.

Thus, there exists a need for improved methods for controlling microbial growth in sugar processing.

### SUMMARY OF THE INVENTION

Considering the primary reason to add lime to the flume system is to suppress microbe proliferation, and also considering the identified challenges associated with managing a high pH flume system, the present invention addresses the foregoing needs by providing improved methods for controlling microbial growth in sugar beet processing. The methods of the invention provide an alternative strategy that is more cost effective.

Sugar processing plants that do not have sufficient liming capacity and/or delivery systems find themselves either (1) not controlling pH or, (2) running relatively low pH, and therefore are viable candidates for the addition of a peroxy acid, such as peracetic acid (PAA), according to the present invention, to effectively control the bacteria as well as pH in the flume system.

Considering the natural pH range of the sugar beet is close to neutral and assuming the depression in flume system pH from that relative neutral position is due to the presence of significant lactic (or related) acids, flume system pH can be increased and furthermore controlled by the addition of a small amount of PAA.

While initially the concept of “adding an acid to increase pH” may be counter-intuitive, one must consider the significant (hundreds of parts per millions) presence of lactic acid being eliminated by a weaker acid (PAA), delivered in much lower concentration (at least ten-fold less). Testing has shown there could be a slight depression to the natural sugar beet and flume system water pH at the onset, but that depression will be relatively minimal (<0.5 pH) and the pH will stabilize instead of continuing to drop.

One version of the method of the invention uses a three-tiered approach as follows: (1) replace the current microbial growth inhibitor (lime) with a true biocide chemistry, i.e., a peroxy acid, such as peracetic acid (PAA); (2) employ genome testing to identify species type and concentration in the water of the flume system, thus allowing more precise and effective treatment; and (3) implement monitoring equipment, including but not limited to oxidation reduction potential (ORP), that validates the treatment schedule and provides for continuous chemical management/adjustment. Optionally, less than the entire amount of lime may be replaced with a true biocide chemistry, i.e., a peroxy acid, such as peracetic acid.

Some advantages of the method of the invention include: (1) more effective management of bacteria through to the sugar extraction plant, by means of using a true biocide vs. relying on pH to suppress proliferation; (2) better control of agent (chemical pump vs. feeder or rotary air lock) and assurance 100% of product introduced to the flume system is available for utilization vs. pebble lime or similar; (3) more neutralized pH will avoid inter-campaign (or more frequent) scale removal costs; (4) more neutralized pH will avoid costs associated with accelerated corrosion; (5) inordinate costs associated with “spiking” lime addition to regain high pH after traditional de-scaling can be avoided; and (6) excessive and accelerated microbial proliferation while deliberately lowering the pH within the “lowering pH gap” with traditional de-scaling can be avoided.

Following a flume system survey, chemistry (e.g., a peroxy acid) can be introduced to the flume system within a designated section (e.g., immediately following the feeder wheel) by way of a metering pump and tote arrangement. Initial usage rates will likely be higher, and steadily reduce to a rate (e.g., <10 ppm) to control microbes and stabilize pH. Continuous feed may be implemented but has been found not to be required in all applications. The flume system water pH will gradually rise from about 4-4.9 to a range from upper 5 to high 6. The overall cost will be less than using lime, and recovery of sugar will improve. Optionally, additional peroxy acid can be added in a recycled water unit and/or water storage pond(s).

In one aspect, the invention provides a method for controlling microbial growth in a sugar processing system. The method comprises (a) adding a peroxy acid into water of an extraction system of the sugar processing system, wherein the extraction system extracts sugar from a sugar-containing plant material, wherein the peroxy acid is added to the extraction system at one or more of the following addition points: i. water entering a diffuser of the extraction system, ii. a mid-tower region of the extraction system, iii. water entering the diffuser from a water supply, iv. pressed pulp water from a pulp press of the extraction system, v. raw juice exiting a cossette mixer of the extraction system, vi. juice recirculated from the diffuser to the cossette mixer, and vii. a cossette slurry entering the cossette mixer.

In another aspect, the invention provides a method for controlling microbial growth in a sugar processing system. The method comprises (a) adding a peroxy acid into water at an addition point of a beet hopper.

In another aspect, the invention provides a method for controlling microbial growth in a sugar processing system, the method comprising: (a) adding a peroxy acid into water at an addition point of a slicing unit that is used to cut beets into cossettes.

In another aspect, the invention provides a method for controlling microbial growth in a sugar processing system, the method comprising: (a) adding a peroxy acid into water at an addition point of a tailing separation unit that is used for recovering chip and tailing material.

In another aspect, the invention provides a method for controlling microbial growth in a sugar processing system, the method comprising: (a) adding a peroxy acid into water at an addition point of a primary clarifier that is used to separate a first process stream from a second process stream.

In another aspect, the invention provides a method for controlling microbial growth in a sugar processing system, the method comprising: (a) adding a peroxy acid at an addition point into water exiting a digester used to separate water from suspended solid materials.

In any of the methods of the invention, step (a) comprises adding the peroxy acid into the addition point as a solution of the peroxy acid, and the solution is essentially free of chelating agents.

In another aspect, the invention provides a method for controlling microbial growth in a sugar processing system, the method comprising: (a) adding a peroxy acid into water at an addition point of a flume system used for transporting a sugar-containing plant material from a delivery or storage location to a wash system, wherein step (a) comprises adding the peroxy acid into the flume system as a solution of the peroxy acid, and the solution is essentially free of chelating agents.

In another aspect, the invention provides a method for controlling microbial growth in a sugar processing system, the method comprising: (a) adding a peroxy acid into water at an addition point of an extraction system of the sugar processing system, wherein the extraction system extracts sugar from a sugar-containing plant material, wherein step (a) comprises adding the peroxy acid at the addition point as a solution of the peroxy acid, and the solution is essentially free of chelating agents.

In any embodiment of the methods, the peroxy acid can have a formula R¹CO₃H, where R¹ is selected from C₁ to C₁₈ alkyl. In any embodiment of the methods, the peroxy acid can have a formula R¹CO₃H, where R¹ is selected from C₁ to C₈ alkyl. In any embodiment of the methods, the peroxy acid comprises peracetic acid.

In any embodiment of the methods, step (a) can comprise reacting a peroxide source with a carboxylic acid to form the peroxy acid. The peroxide source can be hydrogen peroxide, and the carboxylic acid can be acetic acid. In any embodiment of the methods, the peroxide source and the carboxylic acid can be reacted in the water at the addition point.

In any embodiment of the methods, step (a) can comprise adding the peroxy acid into the water at the addition point such that a concentration of the peroxy acid in the water at the addition point is in a range of 1 ppm to 2500 ppm. In any embodiment of the methods, step (a) can comprise adding the peroxy acid into the water at the addition point such that a pH in the water at the addition point is in a range of 2 to 12. In any embodiment of the methods, step (a) can comprise adding the peroxy acid into the water at the addition point such that a pH in the water at the addition point is in a range of 5.5 to 11. In any embodiment of the methods, step (a) can comprise adding the peroxy acid into the water at the addition point such that a pH in the water at the addition point is in a range of 5.5 to 6.9.

In any embodiment of the methods, the method can further comprise (b) determining a concentration of the peroxy acid in the water at the addition point; and (c) adding additional peroxy acid into the water at the addition point when the concentration falls below a predetermined value.

In any embodiment of the methods, the method can further comprise (b) sensing a measurable physical property of the water at the addition point; (c) generating a physical property signal corresponding to the measurable physical property, the physical property signal correlating to a concentration of the peroxy acid in the water at the addition point; (d) transmitting the physical property signal to a controller; and (e) when the concentration falls below a predetermined value stored in the controller, providing a control signal from the controller to open a supply valve in fluid communication with a source of the peroxy acid and the addition point thereby adding additional peroxy acid into the water of the addition point. The measurable physical property can be selected from the group consisting of pH, conductivity, and oxidation reduction potential.

In any embodiment of the methods, the peroxy acid can be added into the water at the addition point as a 1% w/w to 35% w/w aqueous solution of the peroxy acid. In any embodiment of the methods, the peroxy acid can be added into the water at the addition point as a 20% w/w to 30% w/w aqueous solution of the peroxy acid.

In any embodiment of the methods, the sugar-containing plant material is selected from sugar beet, sugar cane, maize, sorghum, carrots, coconuts, nectarines, pineapples, mangoes, jackfruit, peaches, cantaloupe, apricots, bananas, grapes, apples, pears, cherries, oranges, or any combination thereof. The sugar-containing plant material can be sugar beet.

In any embodiment of the methods, the method reduces bacteria count of bacteria that consume sugar. In any embodiment of the methods, the method increases yield of sugar from the sugar processing system. In any embodiment of the methods, the method reduces a count of insects in the water of the flume system.

In any embodiment of the methods, step (a) can comprise sampling the water at the addition point to determine a count of insects in the water at the addition point and adding the peroxy acid into the water at the addition point such that a concentration of the peroxy acid at the addition point reduces the count of insects in the water at the addition point.

In any embodiment of the methods, step (b) can comprise adding additional peroxy acid into water at the addition point. In any embodiment of the methods, step (b) can comprise adding additional peroxy acid into the water at the addition point such that a concentration of the additional peroxy acid at the addition point is in a range of 1 ppm to 2500 ppm. In any embodiment of the methods, step (b) can comprise adding additional peroxy acid into the water at the addition point such that a pH in the water at the addition point is in a range of 2 to 12. In any embodiment of the methods, step (b) can comprise adding additional peroxy acid into the water at the addition point such that a pH in the water at the addition point is in a range of 5.5 to 11.

In any embodiment of the methods, the additional peroxy acid can be added into the water at the addition point as a 1% w/w to 35% w/w aqueous solution of the additional peroxy acid. In any embodiment of the methods, the additional peroxy acid can be added into the water at the addition point as a 20% w/w to 30% w/w aqueous solution of the additional peroxy acid. In any embodiment of the methods, the additional peroxy acid can have a formula R¹CO₃H, where R¹ is selected from C₁ to C₁₈ alkyl. The additional peroxy acid can have a formula R¹CO₃H, where R¹ is selected from C₁ to C₈ alkyl. The additional peroxy acid can comprise peracetic acid.

In any embodiment of the methods, step (b) can comprise reacting a peroxide source with a carboxylic acid to form the additional peroxy acid. The peroxide source can be hydrogen peroxide, and the carboxylic acid can be acetic acid.

These and other features, aspects, and advantages of the present invention will become better understood upon consideration of the following detailed description, drawings, and appended claims.

### BRIEF DESCRIPTION OF THE DRAWINGS

FIG. 1 is a process flow diagram showing part of a prior art beet sugar process.

FIG. 2 is a process flow diagram showing one version of a method according to the invention for controlling microbial growth in sugar processing.

FIG. 3 is a process flow diagram showing another version of a method according to the invention for controlling microbial growth in sugar processing.

FIG. 4 is a process flow diagram showing details of a section of the method shown in FIG. 3.

FIG. 5 is a process flow diagram showing details of another section of the method shown in FIG. 3.

Like reference numerals will be used to refer to like parts from Figure to Figure in the following description of the drawings.

### DETAILED DESCRIPTION OF THE INVENTION

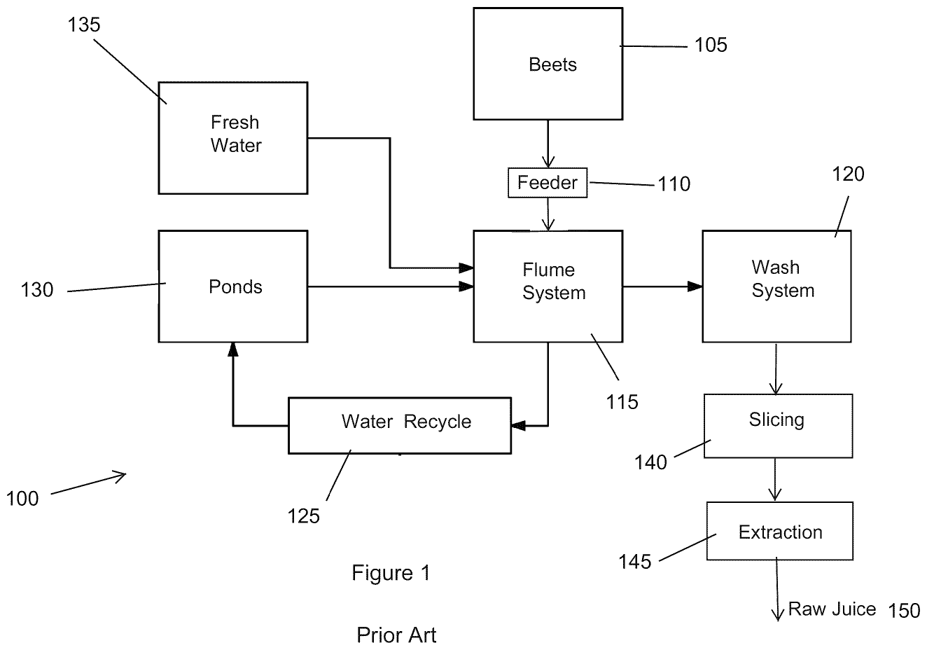

To provide context for the invention, FIG. 1 shows a diagram of an example part of a prior art beet sugar process 100. Beets 105 from a post-harvest delivery or storage location are transported by a feeder 110, typically a feeder wheel, into a water-containing channel of a flume system 115. A non-limiting example water channel may be 100 feet to 200 feet long and have a transverse cross-section of 3-4 square feet. The water channel of the flume system transports the beets into a beet wash system 120 while simultaneously removing field dirt that might adversely affect slicing and extraction. Water from the flume system is recycled via a water recycle conduit 125 and is then stored in one or more water storage ponds 130, which in a non-limiting example embodiment may be 3-5 acre open air ponds. Some of the water from the pond(s) can be later re-used in the flume system together with optional fresh make-up water 135 for subsequent transport of additional beets. The ponds are typically stagnant water that promotes microbial growth in the absence of chemical treatment. As noted above, acid production is a natural, continuous process in flume-water due to acid-forming bacteria activity. To maintain an acceptable flume water pH in the prior art system of FIG. 1, these acids are neutralized by a prior art alkaline additive, such as lime, to maintain the pH in the alkaline range. Maintaining this high pH controls acid forming bacterial activity.

The wash system washes the beets to remove soil and other external contaminants, and the beets are transported to a mechanical slicing unit 140 that can be used to cut each individual sugar beet into a plurality of thin strips known as “cossettes.” The cossettes are then transported to an extraction system 145. Many different machines may be used in the extraction system. The extraction system can comprise placing the cossettes in contact with a counter-current flow of heated water in order to cause the diffusion of sugar-containing materials from the cossettes into the water. The extracted sugar-containing solution exiting the extraction system is referred to as raw juice 150. The raw juice product may be passed through a physical separation apparatus to remove beet juice particles and other suspended solid materials therefrom before further processing of the raw juice. The raw juice can next be purified before sugar crystal production. After the purification, the thin juice is concentrated in an evaporator to provide thick juice. The thick juice is further concentrated by boiling under conditions that allow for crystallization of the sugar.

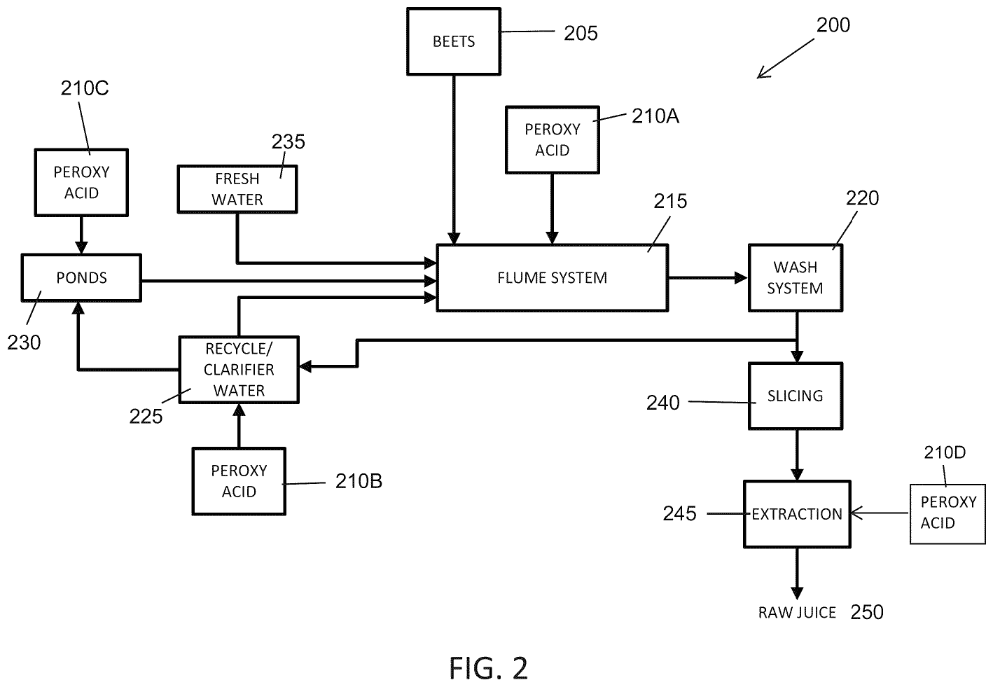

Referring now to FIG. 2, there is shown a process flow diagram showing one version of a method 200 according to the invention for controlling microbial growth in sugar processing. In the closed system method 200 of FIG. 2, beets 205 from a post-harvest delivery or storage location are regulated/metered/controlled by a feeder, typically a feeder wheel, into a water-containing channel of a flume system 215. The water channel of the flume system 215 transports the beets 205 into a beet wash system 220. Water from the wash system 220 is recycled to a water recycle/clarifier water unit 225 which may comprise a mechanical and/or chemical settling system. A coagulant may be added to the water recycle/clarifier water unit 225. The treated water from the water recycle/clarifier water unit 225 is transported for storage in one or more water storage ponds 230. Some of the water from the pond(s) 230 can be later re-used in the flume system 215 together with optional treated water from the water recycle/clarifier water unit 225 and optional fresh make-up water 235 for subsequent transport of additional beets 205.

The wash system 220 provides beets to a slicing unit 240 and an extraction system 245 that produces the raw juice 250. The wash system 220 washes the beets to remove soil and other external contaminants, and the beets are transported to the mechanical slicing unit 240 that can be used to cut each individual sugar beet into a plurality of thin strips known as “cossettes.” The cossettes are then transported to the extraction system 245. Many different machines may be used in the extraction system 245. The extraction system 245 can comprise placing the cossettes in contact with a counter-current flow of heated water in order to cause the diffusion of sugar-containing materials from the cossettes into the water. The extracted sugar-containing solution exiting the extraction system is referred to as the raw juice 250. The raw juice product may be passed through a physical separation apparatus to remove beet juice particles and other suspended solid materials therefrom before further processing of the raw juice. The raw juice can next be purified before sugar crystal production. After the purification, the thin juice is concentrated in an evaporator to provide thick juice. The thick juice is further concentrated by boiling under conditions that allow for crystallization of the sugar.

In the method 200 of FIG. 2, water in the flume system 215 can be treated with a peroxy acid 210A. The peroxy acid 210A can be added into the water of the flume system 215 at many different addition points. As one non-limiting example, the peroxy acid 210A can be added at a point in the water channel after a beet feeder (e.g., feeder wheel) that is positioned between the delivery or storage location and a water channel of the flume system 215. The beet feeder is a rotating device, similar to an old steamboat's paddle wheel, that is affixed to a structure that allows partial immersion of the beet feeder in the flume and meters the beets as they enter the flume. The beet feeder is typically located near the front of the water flume system, after the beet dump and before weed/rock removal equipment.

In the method 200 of FIG. 2, water in the water recycle/clarifier water unit 225 can be treated with a peroxy acid 210B. In the method 200 of FIG. 2, water in the ponds 230 can be treated with a peroxy acid 210C. In the method 200 of FIG. 2, water in the extraction system 245 can be treated with a peroxy acid 210D.

The treatment of the water in the flume system 215 with peroxy acid 210A, and/or the treatment of the water in the water recycle/clarifier water unit 225 with peroxy acid 210B, and/or the treatment of the water in the ponds 230 with peroxy acid 210C, and/or the treatment of the water in the extraction system 245 with peroxy acid 210D can be continuous, substantially continuous, intermittent, cyclic, batch, or any combination thereof. Treatment can be repeated any desired number of times and treatments can be separated by constant or variable time periods. The rate of addition of a peroxy acid 210A, 210B, 210C, 210D can be constant or variable. A peroxy acid 210A, 210B, 210C, 210D can be added in any manner to the water in the flume system 215, and/or the water recycle/clarifier water unit 225, and/or the pond(s) 230, and/or the extraction system 245, for example, by pouring, by nozzle, by spraying, by misting, by curtain, by weir, by fountain, by percolation, by mixing, by injection, or by any combination thereof.

The peroxy acids 210A, 210B, 210C, 210D used in the method 200 of the invention may be an aqueous solution of a peroxy acid which is ready for use. The peroxy acids 210A, 210B, 210C, 210D can have a formula R¹CO₃H, where R¹ is selected from C₁ to C₁₈ alkyl, or the peroxy acids 210A, 210B, 210C, 210D can have a formula R¹CO₃H, where R¹ is selected from C₁ to C₈ alkyl. In one non-limiting example embodiment, the peroxy acids 210A, 210B, 210C, 210D each comprise peracetic acid. Each of the peroxy acids 210A, 210B, 210C, 210D may have the same or different formulas, and each of the peroxy acids 210A, 210B, 210C, 210D may be of the same concentration or different concentrations.

In one version of the method 200, the peroxy acid 210A is added into the water of the flume system 215 as a 1% w/w to 35% w/w aqueous solution of the peroxy acid. In another version of the method 200, the peroxy acid 210A is added into the water of the flume system 215 as a 20% w/w to 30% w/w aqueous solution of the peroxy acid 210A.

In one version of the method 200, the peroxy acid 210B is added into the water of the water recycle/clarifier water unit 225 as a 1% w/w to 35% w/w aqueous solution of the peroxy acid 210B. In another version of the method 200, the peroxy acid 210B is added into the water of the water recycle/clarifier water unit 225 as a 20% w/w to 30% w/w aqueous solution of the peroxy acid 210B.

In one version of the method 200, the peroxy acid 210C is added into the water of the pond(s) 230 as a 1% w/w to 35% w/w aqueous solution of the peroxy acid 210C. In another version of the method 200, the peroxy acid 210C is added into the water of the pond(s) 230 as a 20% w/w to 30% w/w aqueous solution of the peroxy acid 210C.

In one version of the method 200, the peroxy acid 210D is added into the water of the extraction system 245 as a 1% w/w to 35% w/w aqueous solution of the peroxy acid 210C. In another version of the method 200 the peroxy acid 210D is added into the water of the extraction system 245 as a 20% w/w to 30% w/w aqueous solution of the peroxy acid 210C.

Alternatively, the peroxy acids 210A, 210B, 210C, 210D may be prepared by mixing a peroxide source, such as hydrogen peroxide, and an acid which is a precursor of a chosen peroxy acid. The mixing may occur before each of the peroxy acids 210A, 210B, 210C, 210D is added into the water of the flume system 215, and/or the water recycle/clarifier water unit 225, and/or the pond(s) 230, and/or the extraction system 245, respectively; or the mixing may occur after a peroxide source, such as hydrogen peroxide, and a precursor acid which is a precursor of each of the peroxy acids 210A, 210B, 210C, 210D are added into the water of the water of the flume system 215, and/or the water recycle/clarifier water unit 225, and/or the pond(s) 230, and/or the extraction system 245, respectively. For example, each of the peroxy acids 210A, 210B, 210C, 210D may be prepared by reacting a peroxide source with a carboxylic acid to form the peroxy acid. The peroxide source and the carboxylic acid may be reacted in the water of the flume system 215, and/or the water of the water recycle/clarifier water unit 225, and/or the water of the pond(s) 230, and/or the water in the extraction system 245, respectively. In one non-limiting example embodiment, the peroxide source is hydrogen peroxide, and the carboxylic acid is acetic acid.

In one version of the method 200, the peroxy acid 210A is added into the water of the flume system 215 such that a concentration of the peroxy acid 210A in the water of the flume system 215 is in a range of 1 ppm to 2500 ppm. In one non-limiting example version of the method 200, the peroxy acid 210A is added into the water of the flume system 215 such that a pH in the water of the flume system 215 is in a range of 2 to 12. In another non-limiting example version of the method 200, the peroxy acid 210A is added into the water of the flume system 215 such that a pH in the water of the flume system 215 is in a range of 5.5 to 11. In another non-limiting example version of the method 200, the peroxy acid 210A is added into the water of the flume system 215 such that a pH in the water of the flume system 215 is in a range of 5.5 to 6.9, or in a range of 6.3 to 6.9.

In one version of the method 200, the peroxy acid 210B is added into the water of the water recycle/clarifier water unit 225 such that a concentration of the peroxy acid 210B in the water of the water recycle/clarifier water unit 225 is in a range of 1 ppm to 2500 ppm. In one non-limiting example version of the method 200, the peroxy acid 210B is added into the water of the water recycle/clarifier water unit 225 such that a pH in the water of the water recycle/clarifier water unit 225 is in a range of 2 to 12. In another non-limiting example version of the method 200, the peroxy acid 210B is added into the water of the water recycle/clarifier water unit 225 such that a pH in the water of the water recycle/clarifier water unit 225 is in a range of 5.5 to 11. In another non-limiting example version of the method 200, the peroxy acid 210B is added into the water of the water recycle/clarifier water unit 225 such that a pH in the water of the water recycle/clarifier water unit 225 is in a range of 5.5 to 6.9, or in a range of 6.3 to 6.9.

In one version of the method 200, the peroxy acid 210C is added into the water of the ponds(s) 230 such that a concentration of the peroxy acid 210C in the water of the ponds(s) 230 is in a range of 1 ppm to 2500 ppm. In one non-limiting example version of the method 200, the peroxy acid 210C is added into the water of ponds(s) 230 such that a pH in the water of the ponds(s) 230 is in a range of 2 to 12. In another non-limiting example version of the method 200, the peroxy acid 210C is added into the water of the ponds(s) 230 such that a pH in the water of the ponds(s) 230 is in a range of 5.5 to 11. In another non-limiting example version of the method 200, the peroxy acid 210C is added into the water of the ponds(s) 230 such that a pH in the water of the ponds(s) 230 is in a range of 5.5 to 6.9, or in a range of 6.3 to 6.9.

In one version of the method 200, the peroxy acid 210D is added into the water of the extraction system 245 such that a concentration of the peroxy acid 210D in the water of the extraction system 245 is in a range of 1 ppm to 2500 ppm. In one non-limiting example version of the method 200, the peroxy acid 210D is added into the water of the extraction system 245 such that a pH in the water of the extraction system 245 is in a range of 2 to 12. In another non-limiting example version of the method 200, the peroxy acid 210D is added into the water of the extraction system 245 such that a pH in the water of the extraction system 245 is in a range of 5.5 to 11. In another non-limiting example version of the method 200, the peroxy acid 210D is added into the water of the extraction system 245 such that a pH in the water of the extraction system 245 is in a range of 5.5 to 6.9, or in a range of 6.3 to 6.9.

Automated control of the addition of each of the peroxy acids 210A, 210B, 210C, 210D to the water of the flume system 215, and/or the water recycle/clarifier water unit 225, and/or the pond(s) 230, and/or the extraction system 245, respectively, is also possible. A sensor can be placed in each of the water of the flume system 215, and/or the water recycle/clarifier water unit 225, and/or the pond(s) 230, and/or the extraction system 245, respectively, such that fluids passing through the water of the flume system 215, the water recycle/clarifier water unit 225, and/or the pond(s) 230 and/or the extraction system 245 contact the sensor. The sensor measures a physical property of the fluids passing through the water. As used herein, a physical property or a measurable physical property is a property of matter that can be measured or observed without resulting in a change in the composition and identity of a substance. Non-limiting examples of physical properties that can be measured in the sensor include pH, conductivity, oxidation reduction potential, concentration, and density. Sensors are commercially available for measuring these physical properties of the fluids passing through the water channel.

It is contemplated that direct feedback from the sensor can be sent to a programmable logic controller to provide opening and closing times for various valves that control addition of each of the peroxy acids 210A, 210B, 210C, 210D to the water of the flume system 215, and/or the water recycle/clarifier water unit 225, and/or the pond(s) 230 and/or the extraction system 245, respectively. For example, in one version of the method of the invention, the controller can determine a concentration of each of the peroxy acids 210A, 210B, 210C, 210D in the water of the flume system 215, and/or the water recycle/clarifier water unit 225, and/or the pond(s) 230 and/or the extraction system 245, respectively using signal(s) from the sensor, and additional peroxy acid can be added into the water of the flume system 215, and/or the water recycle/clarifier water unit 225, and/or the pond(s) 230, and/or the extraction system 245, respectively by opening a valve when the concentration falls below a predetermined value. In another version of the method of the invention, the sensor is used to sense a measurable physical property (e.g., pH, conductivity, and oxidation reduction potential) of the water of the flume system 215, and/or the water recycle/clarifier water unit 225, and/or the pond(s) 230, and/or the extraction system 245, respectively; the sensor generates a physical property signal corresponding to the measurable physical property wherein the physical property signal correlates to a concentration of the peroxy acid in the water of the flume system 215, and/or the water recycle/clarifier water unit 225, and/or the pond(s) 230, and/or the extraction system 245, respectively; the sensor transmits the physical property signal to the controller; and when the concentration falls below a predetermined value stored in the controller, the controller provides a control signal to open a supply valve in fluid communication with a source of each of the peroxy acids 210A, 210B, 210C, 210D and the water of the flume system 215, and/or the water recycle/clarifier water unit 225, and/or the pond(s) 230, and/or the extraction system 245, respectively thereby adding additional peroxy acid into the water of the flume system 215, and/or the water recycle/clarifier water unit 225, and/or the pond(s) 230, and/or the extraction system 245, respectively.

The method of the invention reduces bacteria count of bacteria that consume sugar. Thus, the method increases yield of sugar from the sugar processing system. Also, the method can reduce a count of insects in the water of the flume system 215, and/or the water recycle/clarifier water unit 225, and/or the pond(s) 230, and/or the extraction system 245. The water of the flume system 215, and/or the water recycle/clarifier water unit 225, and/or the pond(s) 230, and/or the extraction system 245 can be sampled to determine a count of insects and the species of insects in the water, and one can add each of the peroxy acids 210A, 210B, 210C, 210D into the water of the flume system 215, and/or the water recycle/clarifier water unit 225, or the pond(s) 230, and/or the extraction system 245, respectively such that a concentration of each of the peroxy acids 210A, 210B, 210C, 210D in the water of the flume system 215, and/or the water recycle/clarifier water unit 225, and/or the pond(s) 230, and/or the extraction system 245, respectively reduces the count of insects in the water of the flume system 215, and/or the water recycle/clarifier water unit 225, and/or the pond(s) 230, and/or the extraction system 245, respectively.

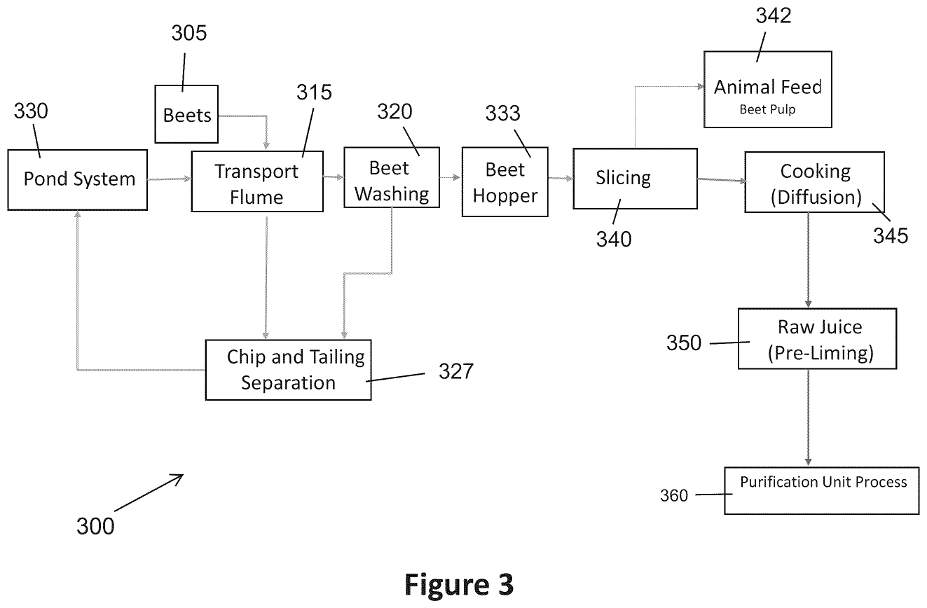

Referring now to FIGS. 3, 4, and 5, there are shown process flow diagrams detailing another version of a closed system method 300 according to the invention for controlling microbial growth in sugar processing. As shown in FIG. 3, beets 305 from a post-harvest delivery or storage location are regulated/metered/controlled by a feeder, typically a feeder wheel, into a water-containing channel of a transport flume system 315. The water channel of the flume system 315 transports the beets 305 into a beet wash system 320. Chips, tailings, and water from the flume system 315 and/or the wash system 320 are recycled to a chip and tailing separation unit 327 which may comprise a mechanical and/or chemical settling system. A coagulant may be added to the chip and tailing separation unit 327. The treated water from the chip and tailing separation unit 327 is transported for storage in a pond system including one or more water storage ponds 330. Some of the water from the pond(s) 330 can be later re-used in the flume system 315 together with optional treated water from the chip and tailing separation unit 327 and optional fresh make-up water for subsequent transport of additional beets 305.

The wash system 320 provides beets to a slicing unit 340 and a cooking/extraction system 345 that produces the raw juice 350. The wash system 320 washes the beets to remove soil and other external contaminants, and the beets are transported to the mechanical slicing unit 340 that can be used to cut each individual sugar beet into a plurality of thin strips known as “cossettes.” The cossettes are then transported to the cooking/extraction system 345 which will be detailed further below. Many different machines may be used in the cooking/extraction system 345. The cooking/extraction system 345 can comprise placing the cossettes in contact with a counter-current flow of heated water in order to cause the diffusion of sugar-containing materials from the cossettes into the water. The extracted sugar-containing solution exiting the extraction system is referred to as the raw juice 350. The raw juice product may be passed through a physical separation apparatus to remove beet juice particles and other suspended solid materials therefrom before further processing of the raw juice. The raw juice can next be purified in a purification unit process 360 before sugar crystal production. After the purification, the thin juice is concentrated in an evaporator to provide thick juice. The thick juice is further concentrated by boiling under conditions that allow for crystallization of the sugar.

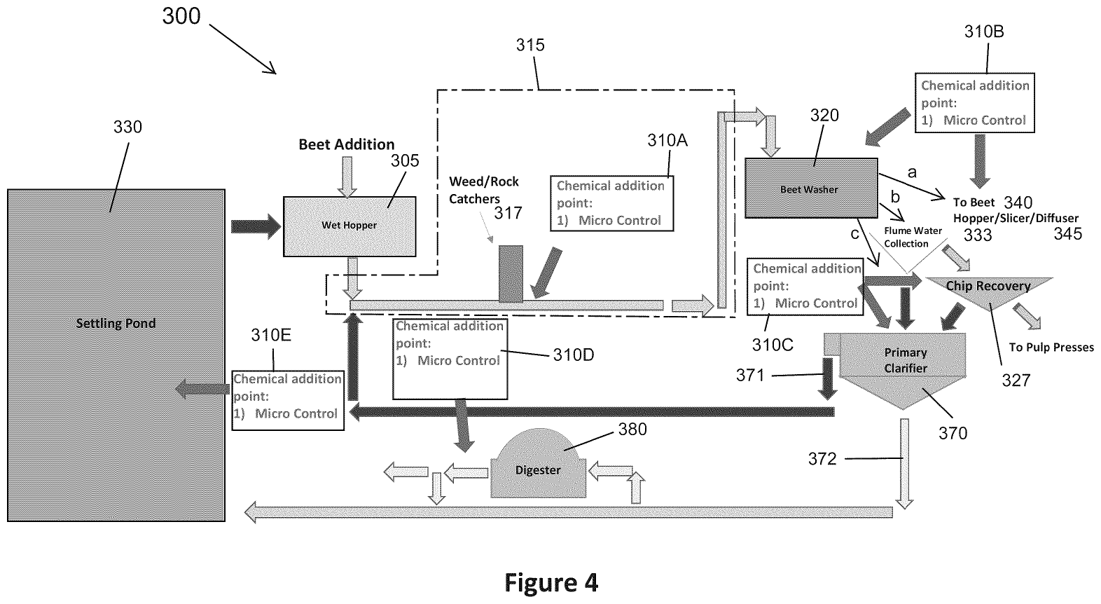

Looking now at FIG. 4, water in the flume system 315 can be treated with a peroxy acid 310A. The peroxy acid 310A can be added into the water of the flume system 315 at many different addition points. As one non-limiting example, the peroxy acid 310A can be added at a point in the water channel after a beet feeder (e.g., feeder wheel) and a weed/rock catcher 317 that is positioned between the delivery or storage location and a water channel of the flume system 315. The beet feeder is a rotating device, similar to an old steamboat's paddle wheel, that is affixed to a structure that allows partial immersion of the beet feeder in the flume and meters the beets as they enter the flume. The beet feeder is typically located near the front of the water flume system, after the beet dump and before weed/rock removal equipment. In the method 300 of FIG. 4, water in the beet washer 320 can be treated with a peroxy acid 310B, which can be used at any point in the beet washer 320 unit block. There are several ways beets get washed and transported to the next step (i.e., slicing), and the peroxy acid 310B can be added at any of the points in the beet washer 320 unit process between the flume system 315 and slicing 340/extraction 345.

Materials in the beet washer 320 can be transported further in the process using a number of process options. In process option a of FIG. 4, larger size materials in the beet washer 320 are transported to a beet hopper 333, then to the slicing unit 340, and then to the cooking/extraction system 345 including a diffuser. In process option b of FIG. 4, medium size materials in the beet washer 320 can be transported to the chip and tailing separation unit 327 for chip recovery. Materials from the chip and tailing separation unit 327 can be transported to pulp presses. In process option c of FIG. 4, water and smaller size suspended solid materials (e.g., mud, plant materials) in the beet washer 320 can be transported to a primary clarifier 370. The primary clarifier 370 separates materials into a first process stream 371 comprising mostly water for recycling to the flume system 315, and a second process stream 372 including smaller size suspended solid materials (e.g., mud, plant materials) which is transported to a digester 380 and then separated water is recycled to the settling pond(s) 330.

In the method 300 of FIG. 4, water in the beet hopper 333 can be treated with a peroxy acid 310B. In the method 300 of FIG. 4, water in the chip and tailing separation unit 327 can be treated with a peroxy acid 310C. In the method 300 of FIG. 4, water in the primary clarifier 370 can be treated with a peroxy acid 310C. In the method 300 of FIG. 4, water exiting the digester 380 can be treated with a peroxy acid 310D. In the method 300 of FIG. 4, water in the settling pond(s) 330 can be treated with a peroxy acid 310E.

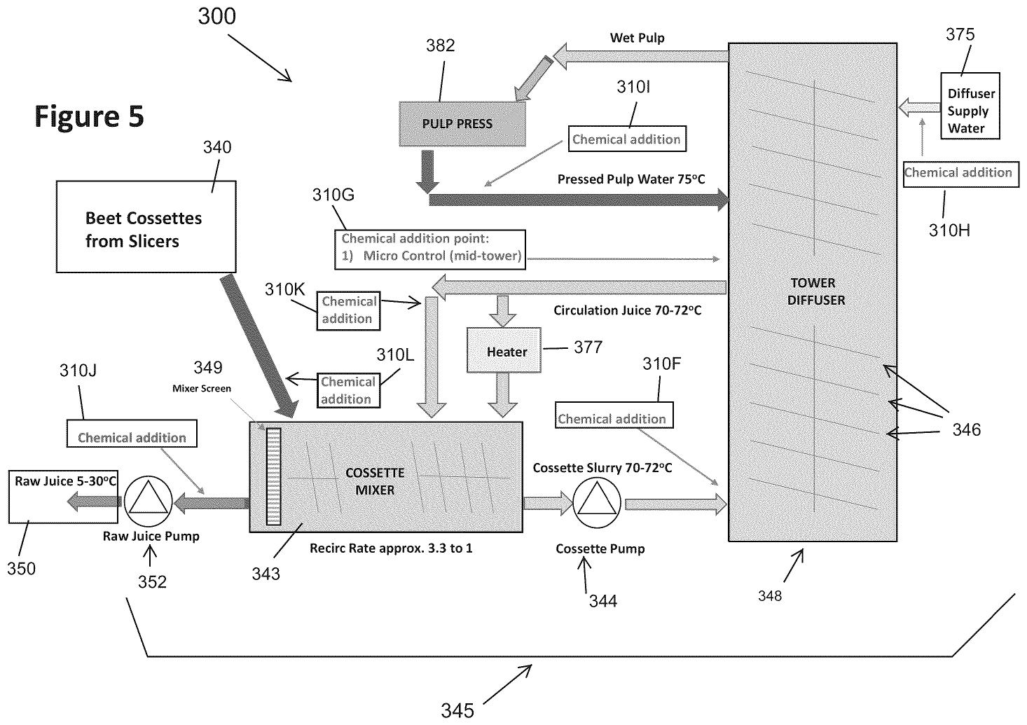

Referring now at FIG. 5, water in the cooking/extraction system 345 can be treated with a peroxy acid. The beet cossettes from the slicer 340 are transported to a cossette mixer 343 of the cooking/extraction system 345, and then a cossette slurry at 70° C.-72° C. is pumped by a cossette pump 344 to a tower diffuser 348 of the cooking/extraction system 345. The mixed cossettes are fed into the tower diffuser 348 at the bottom thereof and the pulp is discharged at the top of the tower diffuser 348. In the method of FIG. 5, water entering the tower diffuser 348 can be treated with a peroxy acid 310F. The tower diffuser 348 includes screens 346 in order to discharge the juice from the inside of the tower diffuser 348. At an upper-tower region of the tower diffuser 348, diffuser water is supplied by a diffuser supply water unit 375 of the cooking/extraction system 345 for continuous countercurrent extraction of juice from the sugar beet cossettes. In the method of FIG. 5, the diffuser water can be treated with a peroxy acid 310H. At an upper-tower region of the tower diffuser 348, wet pulp is transported to a pulp press 382 of the cooking/extraction system 345, and then pressed pulp water is recirculated at 75° C. back to a mid-tower region of the tower diffuser 348. In the method of FIG. 5, the pressed pulp water can be treated with a peroxy acid 310I. The use of peroxy acid augments scalding heaters, or address in cooler areas of circuit, typically found in sections with longer retention. This would include equipment such as the pulp press itself, retention tanks, arc screens, and piping.

In the method of FIG. 5, water in the mid-tower region of the tower diffuser 348 can be treated with a peroxy acid 310G. At a mid-tower region of the tower diffuser 348, circulation juice at 70° C.-72° C. is recirculated back to the cossette mixer 343. A first process stream of the circulation juice can be transported directly back to the cossette mixer 343, and a second process stream of the circulation juice can be transported back to the cossette mixer 343 through a heater 377 of the cooking/extraction system 345 as shown in FIG. 5. In one embodiment, the recirculation rate at the cossette mixer 343 is approximately 3.3 to 1. Raw juice is transported through a mixer screen 349 of the cossette mixer 343, and is pumped by a raw juice pump 352 of the cooking/extraction system 345 to the raw juice unit 350 at 5° C.-30° C. In the method of FIG. 5, raw juice exiting the cossette mixer 343 can be treated with a peroxy acid 310J. In the method of FIG. 5, juice recirculated from the diffuser to the cossette mixer can be treated with a peroxy acid 310K IN the method of FIG. 5, a cossette slurry entering the cossette mixer can be treated with a peroxy acid 310L. Beets and flume water coming into the cutters/cossette mixer can be treated with a peroxy acid. Unless sterile, the beet handling system can inoculate the beets and cossette with further micro contamination. Application directly to the beet washer, or by means of spray-on application at the de-watering table, submersion in trough, tank, or related component, peracetic acid can be used to mitigate infection.

The treatment of the water in the method 300 at the addition points with peroxy acid can be continuous, substantially continuous, intermittent, cyclic, batch, or any combination thereof. Treatment can be repeated any desired number of times and treatments can be separated by constant or variable time periods. The rate of addition of a peroxy acid at addition points 310A, 310B, 310C, 310D, 310E, 310F, 310G, 310H, 310I 310J, 310K, and/or 310L can be constant or variable. A peroxy acid 310A, 310B, 310C, 310D, 310E, 310F, 310G, 310H, 310I, 310J, 310K, and/or 310L can be added in any manner at the addition points, for example, by pouring, by nozzle, by spraying, by misting, by curtain, by weir, by fountain, by percolation, by mixing, by injection, or by any combination thereof.

The peroxy acids 310A, 310B, 310C, 310D, 310E, 310F, 310G, 310H, 310I, 310J, 310K, and/or 310L used in the method 300 of the invention may be an aqueous solution of a peroxy acid which is ready for use. The peroxy acids 310A, 310B, 310C, 310D, 310E, 310F, 310G, 310H, 310I, 310J, 310K, and/or 310L can have a formula R¹CO₃H, where R¹ is selected from C₁ to C₁₈ alkyl, or the peroxy acids 310A, 310B, 310C, 310D, 310E, 310F, 310G, 310H, 310I, 310J, 310K, and/or 310L can have a formula R¹CO₃H, where R¹ is selected from C₁ to C₈ alkyl. Each of the peroxy acids 310A, 310B, 310C, 310D, 310E, 310F, 310G, 310H, 310I, 310J, 310K, and/or 310L may have the same or different formulas, and each of the peroxy acids 310A, 310B, 310C, 310D, 310E, 310F, 310G, 310H, 310I, 310J, 310K, and/or 310L may be of the same concentration or different concentrations.

In one non-limiting example embodiment, the peroxy acids 310A, 310B, 310C, 310D, 310E, 310F, 310G, 310H, 310I, 310J, 310K, and/or 310L each comprise peracetic acid. Chemically, the term “peracetic acid” describes two substances. “Pure” peracetic acid has the chemical formula CH₃CO₃H. Anhydrous peracetic acid explodes violently upon heating. In contrast, aqueous solutions of peracetic acid as sold commercially are created by combining aqueous mixtures of two substances: acetic acid and hydrogen peroxide. At cool temperatures, acetic acid and hydrogen peroxide react over a few days to form an equilibrium aqueous solution containing peracetic acid, acetic acid and hydrogen peroxide. Adding a mineral acid catalyst accelerates the reaction. Peracetic acid is an unstable oxidizing agent and therefore, conventional peracetic acid solutions contain a chelating agent such as etidronic acid (1-hydroxyethylidene-1, 1-diphosphonic acid or HEDP) or dipicolinic acid (pyridine-2,6-dicarboxylic acid or DPA) to slow the rate of oxidation or decomposition of the peracetic acid. However, in the method of the invention, the peroxy acid (e.g., peracetic acid) can be essentially free of chelating agents. As used herein, “essentially free of chelating agents” means that chelating agents are not added to the reactants used to prepare the aqueous solutions of peracetic acid, but chelating agents may be present as an impurity or undesired contaminant in the solution. By using aqueous solutions of peracetic acid essentially free of chelating agents, the method of the invention does not introduce undesirable chelating compounds into the raw juice.

In one version of the method 300, the peroxy acid 310A, 310B, 310C, 310D, 310E, 310F, 310G, 310H, 310I, 310J, 310K, and/or 310L is added at the addition points as a 20% w/w to 30% w/w aqueous solution of the peroxy acid. In one version of the method 300, the peroxy acid 310A, 310B, 310C, 310D, 310E, 310F, 310G, 310H, 310I, 310J, 310K, and/or 310L is added at the addition points as a 1% w/w to 35% w/w aqueous solution of the peroxy acid. In one version of the method, the peracetic acid is added to the system as an aqueous solution including peracetic acid, acetic acid and hydrogen peroxide wherein the peracetic acid is present at 1 wt. % to 35 wt. % in the aqueous solution including peracetic acid, acetic acid and hydrogen peroxide. In another version of the method, the peracetic acid is added to the system as an aqueous solution including peracetic acid, acetic acid and hydrogen peroxide wherein the peracetic acid is present at 1 wt. % to 25 wt. % in the aqueous solution including peracetic acid, acetic acid and hydrogen peroxide. In another version of the method, the peracetic acid is added to the system as an aqueous solution including peracetic acid, acetic acid and hydrogen peroxide wherein the peracetic acid is present at 1 wt. % to 15 wt. % in the aqueous solution including peracetic acid, acetic acid and hydrogen peroxide. In another version of the method, the peracetic acid is added to the system as an aqueous solution including peracetic acid, acetic acid and hydrogen peroxide wherein the peracetic acid is present at 1 wt. % to 10 wt. % in the aqueous solution including peracetic acid, acetic acid and hydrogen peroxide. In another version of the method, the peracetic acid is added to the system as an aqueous solution including peracetic acid, acetic acid and hydrogen peroxide wherein the peracetic acid is present at 1 wt. % to 5 wt. % in the aqueous solution including peracetic acid, acetic acid and hydrogen peroxide.

Alternatively, the peroxy acids 310A, 310B, 310C, 310D, 310E, 310F, 310G, 310H, 310I, 310J, 310K, and/or 310L may be prepared by mixing a peroxide source, such as hydrogen peroxide, and an acid which is a precursor of a chosen peroxy acid. The mixing may occur before each of the peroxy acids 310A, 310B, 310C, 310D, 310E, 310F, 310G, 310H, 310I, 310J, 310K, and/or 310L is added at the addition points, respectively; or the mixing may occur after a peroxide source, such as hydrogen peroxide, and a precursor acid which is a precursor of each of the peroxy acids 310A, 310B, 310C, 310D, 310E, 310F, 310G, 310H, 310I, 310J, 310K, and/or 310L is added at the addition points, respectively. For example, each of the peroxy acids 310A, 310B, 310C, 310D, 310E, 310F, 310G, 310H, 310I, 310J, 310K, and/or 310L may be prepared by reacting a peroxide source with a carboxylic acid to form the peroxy acid. The peroxide source and the carboxylic acid may be reacted at the addition points, respectively. In one non-limiting example embodiment, the peroxide source is hydrogen peroxide, and the carboxylic acid is acetic acid.

In one version of the method 300, the peroxy acid 310A, 310B, 310C, 310D, 310E, 310F, 310G, 310H, 310I, 310J, 310K, and/or 310L is added at the addition points such that a concentration of the peroxy acid 310A, 310B, 310C, 310D, 310E, 310F, 310G, 310H, 310I, 310J, 310K, and/or 310L in the water at the addition points is in a range of 1 ppm to 2500 ppm. In one non-limiting example version of the method 300, the peroxy acid 310A, 310B, 310C, 310D, 310E, 310F, 310G, 310H, 310I, 310J, 310K, and/or 310L is added at the addition points such that a pH in the water at the addition points is in a range of 2 to 12. In another non-limiting example version of the method 300, the peroxy acid 310A, 310B, 310C, 310D, 310E, 310F, 310G, 310H, 310I, 310J, 310K, and/or 310L is added at the addition points such that a pH in the water at the addition points is in a range of 5.5 to 11. In another non-limiting example version of the method 300, the peroxy acid 310A, 310B, 310C, 310D, 310E, 310F, 310G, 310H, 310I, 310J, 310K, and/or 310L is added at the addition points such that a pH in the water at the addition points is in a range of 5.5 to 6.9, or in a range of 6.3 to 6.9.

Automated control of the addition of each of the peroxy acids 310A, 310B, 310C, 310D, 310E, 310F, 310G, 310H, 310I, 310J, 310K, and/or 310L at the addition points, respectively, is also possible. A sensor can be placed in each of the water at the addition points such that fluids passing through the water contacts the sensor. The sensor measures a physical property of the fluids passing through the water. As used herein, a physical property or a measurable physical property is a property of matter that can be measured or observed without resulting in a change in the composition and identity of a substance. Non-limiting examples of physical properties that can be measured in the sensor include pH, conductivity, oxidation reduction potential, concentration, and density. Sensors are available for measuring these physical properties of the fluids passing through the water channel.

It is contemplated that direct feedback from the sensor can be sent to a programmable logic controller providing micro control as shown in FIGS. 4 and 5 to provide opening and closing times for various valves that control addition of each of the peroxy acids 310A, 310B, 310C, 310D, 310E, 310F, 310G, 310H, 310I, 310J, 310K, and/or 310L to the water at the addition points, respectively. For example, in one version of the method of the invention, the controller can determine a concentration of each of the peroxy acids 310A, 310B, 310C, 310D, 310E, 310F, 310G, 310H, 310I, 310J, 310K, and/or 310L in the water at the addition points, respectively using signal(s) from the sensor, and additional peroxy acid can be added into the water at the addition points, respectively by opening a valve when the concentration falls below a predetermined value. In another version of the method of the invention, the sensor is used to sense a measurable physical property (e.g., pH, conductivity, and oxidation reduction potential) of the water at the addition points, respectively; the sensor generates a physical property signal corresponding to the measurable physical property wherein the physical property signal correlates to a concentration of the peroxy acid in the water at the addition points, respectively; the sensor transmits the physical property signal to the controller; and when the concentration falls below a predetermined value stored in the controller, the controller provides a control signal to open a supply valve in fluid communication with a source of each of the peroxy acids 310A, 310B, 310C, 310D, 310E, 310F, 310G, 310H, 310I, 310J, 310K, and/or 310L and the water of at the addition points, respectively.

The method of the invention reduces bacteria count of bacteria that consume sugar. Thus, the method increases yield of sugar from the sugar processing system. Also, the method can reduce a count of insects in the water of the sugar processing system.

The water at the addition points of the sugar processing system can be sampled to determine a count of insects and the species of insects in the water, and one can add each of the peroxy acids 310A, 310B, 310C, 310D, 310E, 310F, 310G, 310H, 310I, 310J, 310K, and/or 310L into the water at the addition points of the sugar processing system, respectively such that a concentration of each of the peroxy acids 310A, 310B, 310C, 310D, 310E, 310F, 310G, 310H, 310I, 310J, 310K, and/or 310L in the water at the addition points of the sugar processing system respectively reduces the count of insects in the water of the addition points, respectively.

Thus, the invention provides methods for controlling microbial growth in sugar processing. While a method for controlling microbial growth in a sugar beet processing system is described herein, the sugar-containing plant material may also be selected from sugar cane, maize, sorghum, carrots, coconuts, nectarines, pineapples, mangoes, jackfruit, peaches, cantaloupe, apricots, bananas, grapes, apples, pears, cherries, oranges, or any combination thereof.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A method for controlling microbial growth in a sugar processing system, the method comprising:
(a) adding a peroxy acid into water of an extraction system of the sugar processing system, wherein the extraction system extracts sugar from a sugar-containing plant material, wherein the peroxy acid is added to the extraction system at one or more of the following addition points:
i. water entering a diffuser of the extraction system,
ii. a mid-tower region of the extraction system,
iii. water entering the diffuser from a water supply,
iv. pressed pulp water from a pulp press of the extraction system,
v. raw juice exiting a cossette mixer of the extraction system,
vi. juice recirculated from the diffuser to the cossette mixer, and
vii. a cossette slurry entering the cossette mixer.

2. The method of claim 1, wherein the addition point is the water entering the diffuser of the extraction system.

3. The method of claim 1, wherein the addition point is the water entering the mid-tower region of the extraction system.

4. The method of claim 1, wherein the addition point is the water entering the diffuser from the water supply.

5. The method of claim 1, wherein the addition point is the pressed pulp water from the pulp press of the extraction system.

6. The method of claim 1, wherein the addition point is the raw juice exiting the cossette mixer of the extraction system.

7. The method of claim 1, wherein the addition point is the juice recirculated from the diffuser to the cossette mixer.

8. The method of claim 1, wherein the addition point is the cossette slurry entering the cossette mixer.

9. (canceled)

10. (canceled)

11. (canceled)

12. (canceled)

13. (canceled)

14. (canceled)

15. (canceled)

16. A method for controlling microbial growth in a sugar processing system, the method comprising:
(a) adding a peroxy acid into water at an addition point of a flume system used for transporting a sugar-containing plant material from a delivery or storage location to a wash system, wherein step (a) comprises adding the peroxy acid into the flume system as a solution of the peroxy acid, and
the solution is essentially free of chelating agents.

17. A method for controlling microbial growth in a sugar processing system, the method comprising:
(a) adding a peroxy acid into water at an addition point of an extraction system of the sugar processing system, wherein the extraction system extracts sugar from a sugar-containing plant material, wherein step (a) comprises adding the peroxy acid at the addition point as a solution of the peroxy acid, and
the solution is essentially free of chelating agents.

18. The method of claim 1 wherein:
the peroxy acid has a formula R¹CO₃H, where R¹ is selected from C₁ to C₁₈ alkyl.

19. The method of claim 1 wherein:
the peroxy acid has a formula R¹CO₃H, where R¹ is selected from C₁ to C₈ alkyl.

20. The method of claim 1 wherein:
the peroxy acid comprises peracetic acid.

21. The method of claim 1 wherein:
step (a) comprises reacting a peroxide source with a carboxylic acid to form the peroxy acid.

22. (canceled)

23. (canceled)

24. The method of claim 1 wherein:
step (a) comprises adding the peroxy acid into the water at the addition point such that a concentration of the peroxy acid in the water at the addition point is in a range of 1 ppm to 2500 ppm.

25. (canceled)

26. (canceled)

27. The method of claim 1 wherein:
step (a) comprises adding the peroxy acid into the water at the addition point such that a pH in the water at the addition point is in a range of 5.5 to 6.9.

28. The method of claim 1 further comprising:
(b) determining a concentration of the peroxy acid in the water at the addition point; and
(c) adding additional peroxy acid into the water at the addition point when the concentration falls below a predetermined value.

29. The method of claim 1, further comprising:
(b) sensing a measurable physical property of the water at the addition point;
(c) generating a physical property signal corresponding to the measurable physical property, the physical property signal correlating to a concentration of the peroxy acid in the water at the addition point;
(d) transmitting the physical property signal to a controller; and
(e) when the concentration falls below a predetermined value stored in the controller, providing a control signal from the controller to open a supply valve in fluid communication with a source of the peroxy acid and the addition point thereby adding additional peroxy acid into the water of the addition point.

30. (canceled)

31. The method of claim 1, wherein:
the peroxy acid is added into the water at the addition point as a 1% w/w to 35% w/w aqueous solution of the peroxy acid.

32. (canceled)

33. (canceled)

34. The method of claim 1, wherein:
the sugar-containing plant material is sugar beet.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
